(19) United States
(12) Patent Application Publication
(10) Pub. No.: US 2022/0015768 A1
(43) Pub. Date: Jan. 20, 2022

(54) SINGLE WIRE FOR MEDICAL TREATMENT TOOL, AND MEDICAL TREATMENT TOOL

(71) Applicant: OLYMPUS CORPORATION, Tokyo (JP)

(72) Inventors: Nobuyuki SUDA, Kamiina-gun (JP); Hideyuki ITO, Tama-shi (JP)

(73) Assignee: OLYMPUS CORPORATION, Tokyo (JP)

(21) Appl. No.: 17/488,985

(22) Filed: Sep. 29, 2021

Related U.S. Application Data
(63) Continuation of application No. PCT/JP2020/010434, filed on Mar. 11, 2020.

(30) Foreign Application Priority Data
Apr. 2, 2019 (JP) 2019-070822

(51) Int. Cl. A61B 17/12; C21D 9/52

(52) U.S. Cl. CPC A61B 17/1214; A61B 2017/00526; C21D 9/52

(57) ABSTRACT

A single wire for a medical treatment tool, includes a main body portion formed from a material having an elastic limit stress equal to or more than 1400 MPa, a 0.2% yield strength equal to or mere than 2000 MPa, and a breaking stress equal to or more than 2100 MPa; and a locking portion formed at a distal end portion of the main body portion and being configured to lock the main body portion to the medical, treatment tool.

The present application is a continuation application of PCT International Application No. PCT/JP2020/010434, filed on Mar. 11, 2020, whose priority is claimed on Japanese Patent Application No. 2019-070822, filed on April 2, 2019. The contents of the PCT International Application and the Japanese Patent Application are incorporated herein by reference.

### TECHNICAL FIELD

The present disclosure relates to a single wire for a medical treatment tool and a medical treatment tool.

### BACKGROUND ART

A medical treatment tool is used for treatment with respect to living tissues, such as grasping, peeling, collecting, crushing, hemostasis, and the like. The medical treatment tool according to the present disclosure is referred to a device including a distal end function portion having a mechanism configured to perform the medical treatment with respect to living tissues such as grasping, peeling, collecting, crushing, hemostasis, and the like, an operation portion for a surgeon to operate, and a wire configured to connect the operation portion and the distal end function portion. Accordingly, a guide wire without the distal end function portion and the like is not included in the medical treatment tool disclosed in the present disclosure.

When the surgeon rotates a proximal end portion of the wire connected to the operation portion, it is possible to transmit the rotation to a distal end portion of the wire so as to rotate the distal end function portion to a desired direction. Also, by pressing to pushing the proximal end portion of the wire, the medical treatment tool may operate the position of the distal end function portion in the body and mechanically open or close the distal end function portion.

The distal end function portion may be adapted by a pair of forceps for grasping the living tissues, a clip for clipping the living tissues to perform the hemostasis, a snare wire or a papillotome knife for resecting or performing the peeling operation with respect to the living tissues, a resection mechanism such as an electrical scalpel or the like, a lithotripsy basket for crushing the cholelith and the like in the bile duct as the living tissues, and the like. Since the medical treatment tool is a disposable product, cost reduction is required.

For example, a clip device disclosed in Japanese Patent No. 4805293 includes a clip and an operating wire that directly engages with the clip. In Japanese Patent No. 4805293, it is disclosed that a stranded wire is more preferable to be provided as the operation wire.

### SUMMARY

According to an aspect of the present disclosure, a single wire for a medical treatment tool, includes a main body portion formed from a material having an elastic limit stress equal to or more than 1400 MPa, a 0.2% yield strength equal to or more than 2000 MPa, and a breaking stress equal to or more than 2100 MPa; and a locking portion formed at a distal end portion of the main body portion and being configured to lock the main body portion to the medical treatment tool.

According to the present aspect of the present disclosure, the material forming the main body portion may further have an elastic limit elongation equal to or more than 1.0%, and a breaking elongation equal to or more than 3.0%.

According to the present aspect of the present disclosure, the main body portion may have a diameter equal to or less than 0.5 mm.

According to the present aspect of the present disclosure, the medical treatment tool may be either of a clip, a pair of grasping forceps, a pair of biopsy forceps, or a papillotome knife.

According to the present aspect of the present disclosure, the main body portion of the single wire may be formed by stainless steel that is reformed by at least one of a straightening process and a heat treatment.

According to the present aspect of the present disclosure, the stainless steel may contain chromium in a mass ratio equal to or more than 16% and nickel in a mass ratio equal to or more than 6%.

According to the present aspect of the present disclosure, the stainless steel may be formed from at least one of a group consisting from SUS301, SUS304, and SUS631.

According to the present aspect of the present disclosure, the main body portion of the single wire may be formed by stainless steel that is reformed by a heat treatment after a straightening process.

According to another aspect of the present disclosure, a medical treatment tool includes the single wire for a medical treatment tool according to the above-described aspects of the present disclosure.

According to another aspect of the present disclosure, a single wire for a medical treatment tool including a distal end function portion for performing medical treatment on living tissues, the single wire for a medical treatment tool includes a distal end portion connected to the function portion; and a proximal end portion, wherein the single wire for a medical treatment tool is configured to transmit a rotation of the proximal end portion to the distal end portion, and the single wire is formed from a material having an elastic limit stress equal to or more than 1400 MPa, a 0.2% yield strength equal to or more than 2000 MPa, and a breaking stress equal to or more than 2100 MPa.

According to a further aspect of the present disclosure, a single wire for a medical treatment tool including a distal end function portion for performing medical treatment on living tissues, the single wire for a medical treatment tool includes a distal end portion connected to the function portion, wherein a distal end portion of the single wire for a medical treatment tool is connected to the distal end function portion, and the single wire is formed from a material having an elastic limit stress equal to or more than 1400 MPa, a 0.2% yield strength equal to or more than 2000 MPa, and a breaking stress equal to or more than 2100 MPa.

### BRIEF DESCRIPTION OF DRAWINGS

FIG. 1 is a partial cross-sectional view schematically showing an example of a medical treatment tool according to an embodiment of the present disclosure.

FIG. 2 is a schematic cross-sectional view showing a single wire according to the present embodiment.

FIG. 3 is a schematic plan view showing an experiment apparatus for evaluating the rotation transmission characteristic.

FIG. 4 is a graph showing an experiment result of a single wire of example 1.

FIG. 5 is a graph showing an experiment result of a single wire of example 2.

FIG. 6 is a graph showing an experiment result of a single wire of example 3.

FIG. 7 is a graph showing an experiment result of a single wire of comparison example 1.

FIG. 8 is a graph showing an experiment result of a single wire of example 2.

FIG. 9 is a graph showing an experiment result of a single wire of example 3.

### DESCRIPTION OF EMBODIMENTS

Hereinafter, a single wire for a medical treatment tool and a medical treatment tool according to an embodiment of the present disclosure will be described with reference to the accompanying drawings.

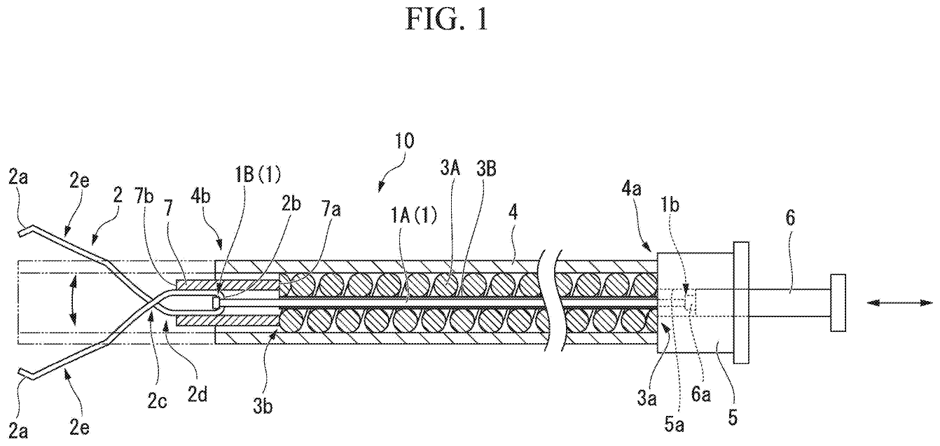

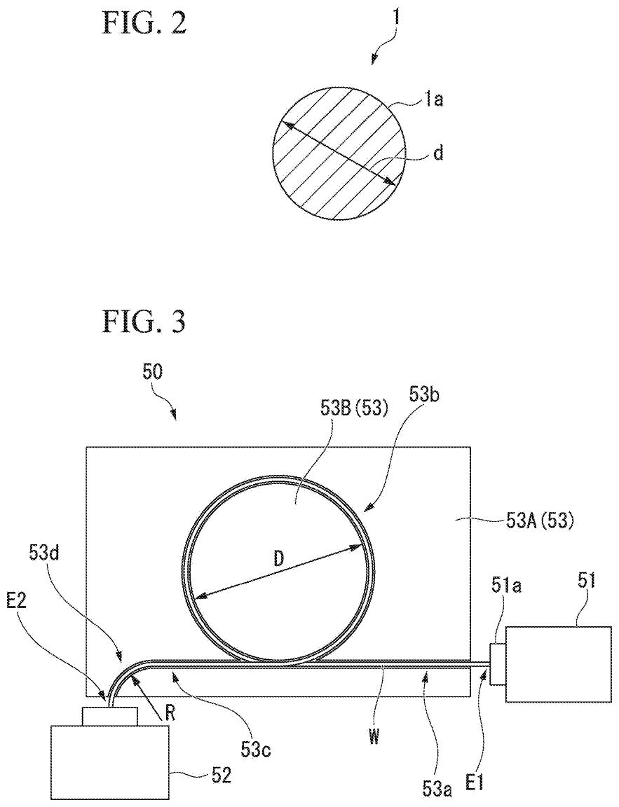

FIG. 1 is a schematic partial cross-sectional view showing an example of a medical treatment tool according to an embodiment of the present disclosure. FIG. 2 is a schematic cross-sectional view showing a single wire according to the embodiment.

As shown in FIG. 1, a treatment tool 10 (medical treatment tool) according to the present embodiment includes a single wire 1 according to the present embodiment. In the example as shown in FIG. 1, the treatment tool 10 is a clip device used by being inserted into a treatment tool channel of an endoscope (not shown). A distal end of the treatment tool 10 is an end portion in a longitudinal direction of the treatment tool 10 and a tip end in an insertion direction with respect to the treatment tool channel. A proximal end of the treatment tool 10 is the end opposite to the distal end in the longitudinal direction of the treatment tool 10.

The treatment tool 10 further includes a clip 2, a tightening ring 7, a coil sheath 3A, an inner sheath 3E, a tube 4, a holder 5, and an operation member 6.

Hereinafter, unless otherwise specified, each component of the treatment tool 10 will be described based on the arrangement in the treatment tool 10. Regarding the end portion of each component of the treatment tool 10 in the longitudinal direction, the end portion being close to the proximal end may be referred to as a tip end portion, and the end portion close to the distal end may be referred to as a base end portion.

The clip 2 is a member configured to grasp the living tissues. The clip 2 is capable of advancing and retreating with respect to a tip end portion of the tube 4, which will be described later, and the clip 2 is capable of performing the grasping operation of the living tissues when the clip 2 is advanced. Further, the clip 2 can be placed in the living body by being separated from the treatment tool 10 in a state of grasping the living tissues.

The configuration of the clip 2 is not particularly limited. In the example shown in FIG. 1, the clip 2 is made of a thin metal strip plate. Hooks 2a where the strip plate is bent are formed at both ends of the strip plate, respectively. The strip plate is bent at a central portion in the longitudinal direction and in a direction such that each hook 2a faces opposite to each other. The bent, portion of the strip plate configures a base end portion 2b of the clip 2. Furthermore, the strips plate intersects once at an intersection portion 2c between each hook 2a and the base end 2b. A substantially elliptical loop portion 2d is formed between the intersection portion 2c and the base end portion 2b. Between the intersection portion 2c and each hook portion 2a, a grasping portion 2e being movable in the opposite direction to each other is formed by the elasticity of the strip plate.

Each grasping portion 2e extends in a V shape from the intersection portion 2c toward each hook portion 2a, and is bent in a direction approaching each other at. an intermediate portion in the longitudinal direction. Each hook 2a projects in a direction opposite to each other.

Although it is not particularly shown in figures, the base end portion 2b is formed with an insertion hole through which a tip end portion of the single wire 1 described below can be locked and can be inserted due to a load equal to or more than a certain value.

As a material of the strip plate configuring the clip 2, for example, a metal material having a spring property, for example, stainless steel, a nickel titanium alloy, a cobalt-chromium alloy, or the like may be used.

The tightening ring 7 is a tubular member having a through hole from a base end portion 7a to a tip end portion 7b. The tightening ring 7 has an inner diameter through which the loop portion 2d and at least a part of the grasping portion 2e of the clip 2 can be inserted.

The tightening ring 7 is used for fixing an opening angle of the clip 2 in a state in which the clip 2 is grasping the living tissues. The tightening ring 7 is configured to fix the grasping portion 2e by a frictional force generated on an inner circumferential surface of the tightening ring 7 when the grasping portion 2e in an open state to grasp the living tissues is retracted into the inside of the tip end portion 7b.

The tightening ring 7 has a length such that the base end portion 2b does not protrude from the base end portion 7a when the clip 2 is fixed.

The material of the tightening ring 7 is not particularly limited as long as the grasping portion 2e can be locked inside. As the material of the tightening ring 7, a resin, metal, or the like having a strength to withstand a reaction force from the clip 2 when the clip 2 is retracted inside and having an elasticity to tighten the clip 2 inward in a radial direction is used.

The tightening ring 7 is arranged closer to the distal end than the clip 2 in a state in which at least a part of the loop portion 2d of the clip 2 is accommodated therein.

The coil sheath 3A is an elongated tubular member made from a tightly wound coil of a metal wire. Since the coil sheath 3A is made from a tightly wound coil, a length thereof is difficult to change even if the coil sheath 3A receives a compressive force in an axial direction (longitudinal direction). The coil sheath 3A has an inner diameter through which an inner sheath 3B described below can be inserted in the axial direction.

The coil sheath 3A has an outer diameter larger than the inner diameter of the tightening ring 7. It is more preferable that an outer diameter of the coil sheath 3A is equal to or larger than the outer diameter of the tightening ring 7.

The coil sheath 3A is arranged at a position substantially coaxial with the tightening ring 7 and at a position closer to the proximal end than the tightening ring 7. The tip portion 3b of the coil sheath 3A can come into contact with the base end portion 7a of the tightening ring 7.

A base end portion 3a of the coil sheath 3A is connected to a holder 5 described below.

The inner sheath 3B is a tubular member arranged along the inner circumferential surface of the coil sheath 3A. The inner sheath 38 has an inner diameter through which the single wire 1 can be slidably inserted. As a material of the inner sheath 38, a resin material having a low frictional force with respect to the single wire 1 is used.

The tube 4 is an elongated tubular member that is capable of accommodating the coil sheath 3A inside. The tube 4 has a flexibility equal to or higher than that of the coil sheath 3A.

The tube 4 has an outer diameter that can be inserted into the treatment tool channel of the endoscope through which the treatment tool 10 is inserted. The tube 4 has an inner diameter through which the coil sheath 3A can be inserted.

A base end portion 4a of the tube 4 is connected to the holder 5 described below.

The tube 4 and the coil sheath 3A can be relatively moved in the longitudinal direction of the treatment tool 10 by operating the holder 5 described below. FIG. 1 shows a state in which the tip end portion 4b of the tube 4 projects toward the proximal end from the tip end portion 3b of the coil sheath 3A. Such a positional relationship is realized by, for example, the coil sheath 3A retracting toward the distal end side or the tube 4 advancing toward the proximal end side by operating the holder 5, which will be described below.

The inner diameter of the tip end portion 4b is equal to or larger than the outer diameter of the tightening ring 7. Accordingly, the tightening ring 7 can be accommodated inside the tip end portion 4b.

It is more preferable that the tube 4 is made of a resin material having a low frictional force with respect to the inner circumferential surface of the treatment tool channel of the endoscope. For example, the material of the tube 4 is mentioned as a polymer resin (synthetic polymer polyamide, high-density/low-density polyethylene, polyester, polytetrafluoroethylene, tetrafluoroethylene-perfluoroalkyl vinyl ether copolymer (PFA), tetrafluoro ethethylene-hexafluoropropylene copolymer, etc.).

The material suitable for the tube 4 is also suitable as the material for the inner sheath 3B described above.

The holder 5 is a member configured to hold the base end portion 3a of the coil sheath 3A and the base end portion 4a of the tube 4 so as to be relatively movable in the longitudinal direction of the treatment tool 10. The holder 5 is arranged outside the endoscope when the treatment tool 10 is used. The surgeon can operate the treatment tool 10 while holding the holder 5.

Inside the holder 5, a hole 5a is provided at a position coaxial with the coil sheath 3A.

The operation member 6 is a rod-shaped member that is slidably inserted into the hole 5a of the holder 5. The operation member 6 can advance and retreat in the axial direction and rotate around a central axis of the hole 5a in the hole 5a.

A fixing portion 6a for fixing a base end portion of the single wire 1 described below is provided at the tip end portion of the operation member 6.

Next, the single wire 2 will be described.

As shown in FIG. 1, the single wire 1 includes a wire main body 1A and a locking portion 1B.

As a cross section perpendicular to the axis shown in FIG. 2, the wire main body 1A has a circular cross section with a diameter d.

The diameter d only has to be a value so as to be insertable through the inner sheath 3B and is not particularly limited. For example, it is more preferable that the diameter d is equal to or less than 0.5 mm. If the diameter d exceeds 0.5 mm, the outer diameter of the tube 4 of the treatment tool 10 becomes large, and it is not suitable to be used in a small-diameter endoscope.

It is further more preferable that the diameter d is equal to or more than 0.3 mm and equal to or less than 0.4 mm.

The wire main body 1A is substantially straight in a natural state in which there is no external force applied thereto. The wire main body 1A is longer than the coil sheath 3A and the inner sheath 3B.

The wire main body 1A is configured to have an elastic limit stress equal to or more than 1400 MPa, a 0.2% yield strength equal to or more than 2000 MPa, and a breaking stress equal to or more than 2100 MPa. In the present embodiment, the elastic limit stress means the limit stress value at which the material is elastically deformed. The 0.2% yield strength means a stress value that causes 0.2% plastic strain in a metal material that does not exhibit a yield phenomenon. The breaking stress means the stress value when the material is broken by an external force.

It is more preferable that the wire main body 1A has an elastic limit elongation equal to or more than 1.0% and a breaking elongation equal to or less than 3.0%.

The material of the wire main body 1A only has to have characteristic values of the elastic limit stress, 0.2% yield strength, and breaking stress in the above-described range and is not particularly limited. For example, examples of the material of the wire main body 1A include stainless steel, nickel titanium alloy, cobalt-chromium alloy and the likes. A surface of the wire main body 1A may be coated with an appropriate metal material for the purpose of improving corrosion resistance, slidability, and the like.

The stainless steel is particularly preferable to be used as the material of the wire main body 1A in that the corrosion resistance is good and the above-described characteristic values can be easily obtained.

For example, when the wire main body 1A is formed from the stainless steel, it is more preferable to contain 16% or more of chromium (Cr) and 6% or more of nickel (Ni).

For example, the stainless steel used for the wire main body 1A is more preferably formed from at least one stainless steel selected from the group consisting of SUS301, SUS304, and SUS631 (see Japanese Industrial Standards JIS, the same applies hereinafter).

As the material of the wire main body 1A, a reformed metal material having the above-mentioned characteristic values may be used.

The example of the reforming means is not particularly limited. As the reforming means, an appropriate reforming means for hardening the metal material is used. For example, at least one of the straightening process and the heat treatment may be used as the reforming means.

Commercially available metal wires have a curl while being wound around a bobbin to be stored such that the metal wires are still bent even after being cut. Accordingly, the metal wires used for the treatment tool are subjected to the straightening process so as to correct the curl.

However, the straightening process to correct the curl is performed for the purpose of making the straightness within a certain range.

Generally, the straightening process for the purpose of correcting curl has almost no reforming effect such that it is considered that a metal wire that does not have the above-mentioned characteristic values before being processed will not have the above-mentioned characteristic values even after being processed.

According to the study of the present inventor, the metal wire can be reformed by adjusting the load during the straightening process. For example, tension load, sliding load, bending load, heat load and the like can be considered to be examples of the load during the straightening process.

The conditions of the straightening process necessary for the reforming means can be experimentally determined according to the type of the metal wire material, the wire diameter, and the like.

The heat treatment used for the reforming means only has to be a heat treatment suitable to harden the metal wire and is not particularly limited. The conditions of the heat treatment for making the metal wire material to satisfy the above-described characteristic value range may be experimentally determined according to the straightening process conditions, the type of the metal wire material, the wire diameter, and the like.

As a result of diligent studies by the present inventor, it is more preferable to use both of the straightening process and the heat treatment as the reforming means. In this case, even if the above-mentioned characteristic value range cannot be obtained when only one of the straightening process and the heat treatment is performed, it is possible to satisfy the above-mentioned characteristic value range by performing both of the straightening process and the heat treatment.

In particular, when the heat treatment is performed after the straightening process, a more superior reforming effect can be obtained.

The locking portion 1B is formed at the tip end portion of the wire main body 1A for the purpose of locking the wire main body 1A to the clip 2, wherein the wire main body 1A is inserted through the insertion hole in the base end portion 2b of the clip 2.

A shape of the locking portion 1B only has to be determined such that the locking portion 1B can be locked in the insertion hole with a force less than a predetermined pull-cut force and the locking portion 1B can be pulled out from the insertion hole with a force equal to or more than the predetermined pull-out force and the shape of the locking portion 1B is not particularly limited. However, the shape of the locking portion 1B is a shape capable of transmitting the traction force and the rotational force transmitted by the single wire 1 when being locked to the clip 2.

The locking portion 1B is configured such that a rotationally asymmetrical shape having a width wider than the outer diameter d of the single wire 1 is used to form at least part of the locking portion 1B. For example, the shape of the locking portion 1B may be a flat shape having the width larger than the outer diameter d.

The locking portion 1B may be formed by deforming the tip end portion of the wire main body 1A, adding a member to the tip end portion of the wire main body 1A, or the like. In a case in which the locking portion 1B is formed by adding a member, the material of the locking portion 1B may be different from that of the wire main body 1A.

Examples of the method for forming the locking portion 1B include pressing, caulking, laser melting, plasma welding, brazing, and the like. The end portion of the single wire 1 opposite to the locking portion 1B is fixed to the fixing portion 6a of the operation member 6. As a result, the single wire 1 rotates interlocking with the rotation of the operation member 6 around the central axis. Furthermore, the single wire 1 advances and retreats interlocking with the advance and retraction of the operation member 6 along the central axis of the operation member 6.

Next, the effect of the treatment tool 10 and the single wire 1 will be described.

Hereinafter, in order to make the description to be easy, an example in which the tube 4 advances arid retracts by operating the holder 3 will be described.

In order for the surgeon to perform the procedure of clipping the living tissue using the treatment tool 10, firstly, the endoscope (not shown) is inserted into the body of the patient.

At this time, the treatment tool 10 is in a state in which the clip 2 is accommodated in the tip end portion 4b of the tube 4. This state is realized by the surgeon moving the tube 4 toward the distal end where the clip 2 is provided (see alternate long and short dash line in FIG. 1). As a result, the treatment tool 10 becomes a linear body having an outer diameter equal to or less than that of the tube 4 except for the configurations at the proximal end side from the holder 5.

The treatment tool 10 is inserted into the treatment tool channel from the distal end with the clip 2 closed by the tube 4.

After the distal end of the treatment tool 10 protrudes outward from the tip end portion of the endoscope, the surgeon advances and retracts the holder 5 in the insertion direction to adjust the distance between the treatment target and the clip 2. Furthermore, the surgeon adjusts the rotation position of the clip 2 by rotating the operation member 6 around the central axis. The rotation of the operation member 6 is transmitted to the base end portion 2b of the clip 2 by the rotation of the single wire 1 interlocking with the rotation of the single wire 1.

After the clip 2 has been rotated to a proper posture, the surgeon pulls the operation member 6 toward the proximal end side. As a result, the clip 2 is retracted into the tightening ring 7, and each grasping portion 2e is closed. As a result, each hook 2a bites into the living tissues.

When each grasping portion 2e is retracted into the tightening ring 7, the reaction force from each grasping portion 2e to the tightening ring 7 increases. Each grasping portion 2e is firmly locked to the inner circumferential surface of the tightening ring 7 by the frictional force.

Furthermore, when the surgeon retracts the operation member 6, the tightening ring 7 is locked to the tip end portion 3b of the coil sheath 3A such that the single wire 1 is pulled toward the proximal end side. When the traction force applied to the single wire 1 exceeds a certain value, the locking portion 1B is pulled out from the insertion hole of the clip 2. Since the clip 2 and the tightening ring 7 are separated from the single wire 1, they are separated from the treatment tool 10. When the surgeon retracts the treatment tool 10, the clip 2 grasping the living tissues is indwelled in the body of the patient together with the tightening ring 7.

The surgeon removes the treatment tool 10 out of the treatment tool channel to end the procedure.

Here, the effects of the single wire 1 in adjusting the rotation of the clip 2 will be described in detail.

In the process of adjusting the rotation of the clip 2, it is desirable that a rotation angle of the clip 2 matches a rotation angle of the operation member 6.

However, the single wire 1 receives a frictional force by coming into contact with the inner sheath 3B in the coil sheath 3A in a state in which a plurality of bent portions are formed during the actual usage. The work of the torque applied to the operation member 6 is consumed by the work of resisting the frictional force and the torsional deformation of the single wire 1. Until the torsional deformation of the base end portion of the single wire 1 reaches a certain amount (hereinafter referred to as an initial stage), the rotation angle of the clip 2 is smaller than the rotation angle of the operation member 6. Furthermore, in the initial stage, the rotation amount of the clip 2 is non-linear with respect to the rotation amount of the operation member 6 such that it is difficult to adjust the rotation angle of the clip 2.

When the strain amount of the single wire 1 increases to some extent, the rotational torque of the operation member 6 is transmitted to the entire single wire 1 and the entire single wire 1 starts to rotate against the frictional force. At this time, if the frictional force is constant, the rotation increment of the clip 2 corresponds to the rotation increment of the operation member 6.

However, since the frictional resistance received by the single wire 1 varies in the longitudinal direction depending on the bending state or the like, there is a case in which the stick-slip in the rotation direction occurs with respect to the single wire 1. For example, when the rotation is hindered at a part of the single wire 1 due to the frictional force, the strain energy accumulated in the single wire 1 increases, and the strain energy is released when the rotation resumes to bias the single wire 1. As a result, the cumulative rotation amount of the operation member 6 is transmitted to the clip 2 during a short period. As a result, even if the rotation increment of the operation member 6 is constant, the rotation increment of the clip 2 varies.

After the initial stage, unless the rotation is locked or the single wire 1 is damaged, the rotation increment of the clip 2 coincides with the rotation increment of the operation member 6 on average even if the above-described change occurs. Hereinafter, the stage after such initial movement stage is referred to as a steady rotation stage.

In order to improve the operability of the treatment tool 10, it is more preferable that the rotation angle of the operation member 6 from the end of the initial stage to the start of the steady rotation stage is small. That is, it is more preferable that the torsional rigidity of the single wire 1 is as large as possible.

Furthermore, in the steady rotation stage, it is more preferable that the difference between the rotation increment of the operation member 6 and the rotation increment of the clip 2 is small. That is, in the steady rotation stage, it is more preferable that the linearity of the rotation transmission characteristic is superior. It is more preferable that the accumulated amount of strain energy in the steady rotation stage is as small as possible since the rotation change can be easily suppressed even if the stick-slip occurs.

According to the above-described consideration, in order to improve the operability of the treatment tool 10, it is preferable that the single wire 1 has the high torsional rigidity.

The single wire 1 used in a bent state in the treatment tool channel is repeatedly bent by rotating around the central axis in the curved path. Accordingly, it is considered that the flexural rigidity of the single wire 1 is also related to the operability of the treatment tool 10.

Furthermore, a wire with a small diameter such as the single wire 1 may be partially plastically deformed depending on the usage conditions such as the bending amount or the like. In this case, it is considered that the operability of the single wire 1 cannot be evaluated only by the elastic deformation characteristics based on the elastic modulus and the like.

As a result of diligently studying the characteristics required for the single wire based on the above-mentioned viewpoint, the present inventor has newly found the condition of the single wire that improves the operability of the medical treatment tool, and reaches the present invention.

Specifically, it is found that by setting at least the elastic limit stress, 0.2% yield strength, and breaking stress of the wire main body 1A in the single wire 1 within the above-described ranges, the operability in the medical treatment tool is improved.

It is considered that the characteristic values of elastic limit stress, 0.2% yield strength, and breaking stress are related to the improvement of elasticity and toughness of the material.

That is, the elastic limit stress, 0.2% yield strength, and breaking stress are not characteristic values that directly represent the elastic modulus of the material; however, they are characteristic values are related to the elastic modulus in the metal material. Furthermore, since each characteristic value is related to the characteristic of the plastic region, the characteristic values are considered to be suitable for the evaluation of the single wire 1 including the plastic deformation.

Therefore, it is considered that the operability of the medical treatment tool is improved when the characteristic values of the wire main body 1A of the single wire 1 according to the present embodiment are within the above-described range.

Furthermore, when the elastic limit elongation and the breaking elongation in the wire main body 1A of the single wire 1 are set in the above-described ranges, the single wire 1 has higher toughness.

For example, the larger the elastic limit elongation, the larger the deformation in the elastic region is possible.

For example, the ductility is small if the breaking elongation is small such that the plastic deformation is unlikely to occur, or the shape change is small even if the plastic deformation occurs.

According to such a characteristic, it is considered that a light operation becomes possible since the deformation of the single wire 1 that is repeatedly bent in the bent state is smooth.

As described above, according to the present embodiment, since the single wire 1 has the above-described characteristic values, the rotation transmission characteristic from the base end portion toward the tip end portion of the single wire 1 becomes good even the single wire 1 is in the deformed state along the bent coil sheath 3A and the inner sheath 3B in the treatment tool channel. Accordingly, the rotation angle of the operation member 6 is suitably transmitted to the clip 2. As a result, according to the single wire 1, the operability of the treatment tool 10 is improved.

In the description of the above embodiment, an example in which the medical treatment tool is a clip device has been described. However, the medical treatment device according to the present disclosure is not limited to the clip device as long as the single wire can be used. The medical treatment tool according to the present invention may be, for example, a pair of grasping forceps, biopsy forceps, a papillotome knife and the like.

In the description of the above embodiment, the case where the medical treatment tool has one single wire is described. However, a plurality of single wires may be used in the medical procedure as long as the single wires do not form a stranded wire.

In the description of the above embodiment, the case in which the single wire 1 includes the metal wire main body 1A and the surface of the wire main body 1A is not formed with a non-metal coating has been described. However, the single wire may have a non-metal coating on the surface thereof.

### EXAMPLES

Next, examples of the single wire according to the above-described embodiment will be described together with comparison examples. The following [Table 1] shows the configurations and evaluation results of Examples 1 to 3 and Comparison Examples 1 to 3.

TABLE 1

| | | | CHARACTERISTIC VALUE | | | | | |
|---|---|---|---|---|---|---|---|---|
| | | | ELASTIC | 0.2% | | ELASTIC | | |
| | SINGLE WIRE | | CRITICAL | PROOF | BREAKING | CRITICAL | BREAKING | ROTATION |
| | | DIAMETER | STRESS | STRESS | STRESS | ELONGATION | ELONGATION | TRANSMISSION |
| | MATERIAL | (mm) | (MPa ) | (MPa) | (MPa) | (%) | (%) | CHARACTERISTIC |
| EXAMPLE 1 | SUS631J1 | 0.35 | 1425 | 2045 | 2359 | 1.24 | 2.46 | ⊚ |
| EXAMPLE 2 | SUS301 | 0.35 | 1427 | 2043 | 2348 | 1.08 | 2.07 | ⊚ |
| EXAMPLE 3 | SUS304 | 0.35 | 1456 | 2120 | 2728 | 1.13 | 2.78 | ∘ |
| COMPARISON | SUS631J1 | 0.35 | 1367 | 1822 | 2220 | 1.16 | 3.37 | x |
| EXAMPLE 1 | | | | | | | | |
| COMPARISON | SUS301 | 0.35 | 1442 | 1894 | 2351 | 1.25 | 3.21 | x |
| EXAMPLE 2 | | | | | | | | |
| COMPARISON | SUS304 | 0.35 | 1104 | 1485 | 1964 | 0.98 | 5.06 | x |
| EXAMPLE 3 | | | | | | | | |

### Example 1

Example 1 is an example corresponding to the single wire 1 according to the present embodiment.

As shown in [Table 1], SUS631J1 having a diameter of 0.35 mm (see Japanese Industrial Standards JIS, the same applies hereinafter) was used as the material of the single wire 1 of Example 1 (shown as “single wire” in [Table 1]). The material of SUS631J1 is a stainless steel containing 16% or more of Cr and 6.5% or more of Ni, and about 1.0% of aluminum (Al) is added therein. SUS631J1 is a steel type included in SUS631.

Since the wire was wound around a bobbin, it is necessary to perform the straightening process in order to obtain the straightness.

The wire was cut after being subjected to the straightening process. During the straightening process, the load was adjusted.

After the straightening process in which the load was adjusted, the wire was processed by an age-hardened heat treatment at 470 degrees Celsius or higher to improve the elastic marginal stress, the 0.2% yield strength, and the breaking stress.

In this manner, by modifying the commercially available wire, the wire main body 1A of Example 1 was manufactured.

The wire main body 1A was cut to form a test piece for measuring the characteristic values and a single wire for evaluating the rotation transmission characteristics.

A length of the test piece for measuring the characteristic value was 150 mm.

A length of the single wire for evaluating the rotation transmission characteristic was 2500 mm.

Furthermore, a single wire 1 for a treatment tool was formed from the wire main body 1A. In the single wire 1 for the treatment tool, after the wire main body 1A was cut to 2300 mm, the locking portion 1B was formed at the tip end portion by brazing.

As the characteristic values of the test piece, the elastic limit stress, the 0.2% yield strength, the breaking stress, the elastic limit elongation, and the breaking elongation were determined from a stress strain curve obtained during a tension test by using the precision universal testing machine Autograph (registered trademark) AG-plus (product name; manufactured by Shimadzu Co., Ltd.) However, the 0.05% yield strength was used as the elastic limit stress.

In the tensile test, a 5 kN load cell was used. The grasping distance of the test piece was set to 50 mm. An air chuck was used as the chuck method for the test piece. The test speed was 1 mm/min.

As shown in [Table 1], the result of the above test was that the elastic limit stress of the single wire 1 of Example 1 was 1425 MPa, the 0.2% yield strength was 2045 MPa, the breaking stress was 2359 MPa, the elastic limit elongation was 1.24%, and the breaking elongation was 2.46%.

### Example 2

The single wire 1 of Example 2 was the same as that of Example 1 except that SUS301 was used as the material thereof and the reforming conditions were changed accordingly.

SUS301 is a stainless steel containing 16% or more of Cr and 6% or more of Ni. The wire of SUS301 was heat-treated at 300 degrees Celsius or higher after the straightening process in which the load was adjusted so as to improve the elastic limit stress, the 0.2% yield strength, and the breaking stress.

By reforming the commercially available wire in this manner, the wire main body 1A of Example 2 was manufactured.

From the wire main body 1A of Example 2, a test piece for measuring characteristic values, a single wire for evaluating rotation transmission characteristics, and a single wire 1 for treatment tool were manufactured in the same manner as that in Example 1.

As a result of the same test as in Example 1, the elastic limit stress of the single wire 1 of Example 2 was 1427 MPa, the 0.2% yield strength was 2043 MPa, the breaking stress was 2348 MPa, the elastic limit, elongation was 1.08%, and the breaking elongation was 2.07%.

### Example 3

The single wire 1 of Example 3 was the same as that of Example 1 except that SUS304 was used as a material and the reforming conditions are changed accordingly.

SUS304 is a stainless steel containing 13% or more of Cr and 8% or more of Ni. The wire of SUS304 was heat-treated at 400 degrees Celsius or higher after the straightening process in which the load was adjusted so as to improve the elastic limit stress, the 0.2% yield strength, and the breaking stress.

By reforming the commercially available wire in this manner, the wire main body 1A of Example 3 was manufactured.

From the wire main body 1A of Example 3, a test piece for measuring characteristic values, a single wire for evaluating rotation transmission characteristics, and a single wire 1 for treatment tool were manufactured in the same manner as that in Example 1.

As a result of the same test as that in Example 1, the elastic limit stress of the single wire 1 of Example 3 was 1456 MPa, the 0.2% yield strength was 2120 MPa, the breaking stress was 2728 MPa, the elastic limit elongation was 1.13%, and the breaking elongation was 2.78%.

As described above, the single wires 1 of Examples 1 to 3 had an elastic limit stress equal to or more than 1400 MPa, a 0.2% yield strength equal to or more than 2000 MPa, and a breaking stress equal to or more than 2100 MPa. Furthermore, the single wires 1 of Examples 1 to 3 had an elastic limit elongation equal to or more than 1.0% and a breaking elongation equal to or less than 3.0%.

### Comparison Example 1

The single wire of Comparison Example 1 was the same as that of Example 1 except that the single wire does not have the characteristic values necessary for the single wire according to the present disclosure.

In Comparison Example 1, for the purpose of adjusting the characteristic value, the wire was subjected to the age-hardening heat treatment before the straightening process and then the straightening process was performed.

As a result, the elastic .limit stress of the single wire of Comparison Example 1 was 1367 MPa, the 0.2% yield strength was 1822 MPa, the breaking stress was 2220 MPa, the elastic limit elongation was 1.16%, and the breaking elongation was 3.37%.

In Comparison Example 1, the heat treatment and the straightening process were performed; however, the reforming effect for the characteristic values required for the present disclosure was not achieved except for the breaking stress.

### Comparison Example 2

The single wire of Comparison Example 2 is the same as that of Example 2 except that it does not have the characteristic value required for the single wire according to the present disclosure.

In Comparison Example 2, the straightening process was performed for the purpose of adjusting the characteristic value.

As a result, the elastic limit stress of the single wire of Comparison Example 2 was 1442 MPa, the 0.2% yield strength was 1894 MPa, the breaking stress was 2351 MPa, the elastic limit elongation was 1.25%, and the breaking elongation was 3.21%.

In Comparison Example 2, as a result of the wire being reformed to some extent by the straightening process, the characteristic values of the elastic limit stress and the breaking stress were substantially the same as those in Example 2. However, since the 0.2% yield strength was less than 2000 MPa, the characteristic value required for the present invention was not achieved.

### Comparison Example 3

The single wire of Comparison Example 3 is the same as that of Example 3 except that it does not have the characteristic value required for the single wire according to the present disclosure.

In Comparison Example 3, the straightening process was performed for the purpose of adjusting the characteristic value.

As a result, the elastic limit stress of the single wire of Comparison Example 3 was 1104 MPa, the 0.2% yield strength was 1485 MPa, the breaking stress was 1964 MPa, the elastic limit elongation was 0.98%, and the breaking elongation was 5.06%.

In Comparison Example 3, straightening was performed; however, the reformation to achieve the necessary characteristic values of either of the elastic limit stress, the 0.2% yield strength, and the breaking stress required for the present invention was not obtained.

### Evaluation

The rotation transmission characteristics were evaluated using the single wire for evaluating the rotation transmission characteristics of Examples 1 to 3 and Comparison Examples 1 to 3.

FIG. 3 is a schematic plan view showing a test apparatus for evaluating the rotation transmission characteristics.

As shown in FIG. 3, a test apparatus 50 includes a wire rotation portion 51, a rotation angle detection portion 52, and a wire holder 53.

The wire rotation portion 51 has a grasping portion 51a driven by a motor. The grasping portion 51a grasps the first end portion E1 of the single wire W. The rotation speed of the grasping portion 51a in the present evaluation was set to equal to or less than 90 deg/sec.

The rotation angle detection portion 52 detects the rotation speed of the second end portion E2 on the opposite side to the first end portion E1 of the single wire W. An angle detection sensor was used as the rotation angle detection unit 52.

The wire holder 53 keeps the bent shape of the single wire W constant while the single wire W is rotated. The wire holder 53 includes a flat plate-shaped base 53A and a cylindrical portion 53B fixed on the base 53A. The diameter D of the cylindrical portion 53B was 150 mm, and the height was equal to or more than twice the diameter of the single wire W. The diameter D is set in the range of 100 mm to 200 mm according to the desired product function.

A first groove portion 53a, a second groove portion 53b, a third groove portion 53c, and a fourth groove portion 53d are formed on the surface of the base 53A. The first groove portion 53a, the second groove portion 53b, the third groove portion 53c, and the fourth groove portion 53d are U-shaped grooves having a groove width and a depth for accommodating the single wire W such that the single wire W is slidable therein. However, in FIG. 3, in order to make it easy to view, a gap is provided between the outer shape of the single wire W and the inner circumferential surface of the groove. A resin tube through which the single wire W is insertable is provided in each groove. For example, a tube made from PFA having an inner diameter of φ0.75 mm is an example of the resin tube.

The first groove portion 53a extends straight in the tangential direction of the cylindrical portion 53B.

The second groove portion 53b extends in a circular shape along the outer circumference of the cylindrical portion 53B.

The third groove portion 53c extends in the same straight line as the first groove portion 53a and communicates with the first groove portion 53a and the second groove portion 53b.

The fourth groove portion 53d is a bent groove extending along an arc having a central angle of 90 degrees from an end portion of the third groove portion 53c opposite to the first groove portion 53a. A radius R of the fourth groove 53d was set to 25 mm. A central angle of the bent groove is set in the range of 90 degrees to 130 degrees, and the radius R is set in the range of 15 mm to 45 mm according to the desired product function.

Although not shown in figures, the wire holder 53 further includes a wire retainer for preventing the single wire W from popping out from each groove after arranging the single wire W in each groove.

The single wire W was inserted through the first groove portion 53a, then double-wound around the cylindrical portion 53B along the second groove portion 53b, and then inserted through the third groove portion 53c and the fourth groove portion 53d.

The first end portion E1 of the single wire W protrudes from the first groove portion 53a to the lateral side (right side as shown in the figure) of the base 53A and is grasped by the grasping portion 51a.

The second end portion E2 of the single wire W protrudes from the fourth groove portion 53d to the lateral side (lower side as shown in the drawing) of the base 53A and is inserted into the rotation angle detection portion 52.

The single wire wires of each example and each comparison example were attached to the test apparatus 50 as the above-described single wire W. The rotation angle of the second end portion E2 when the grasping portion 51a was rotated three times (rotation angle 1080 degrees) at the above-described rotation speed was measured.

### Evaluation Results

The evaluation result of the rotation transmission characteristic will be described.

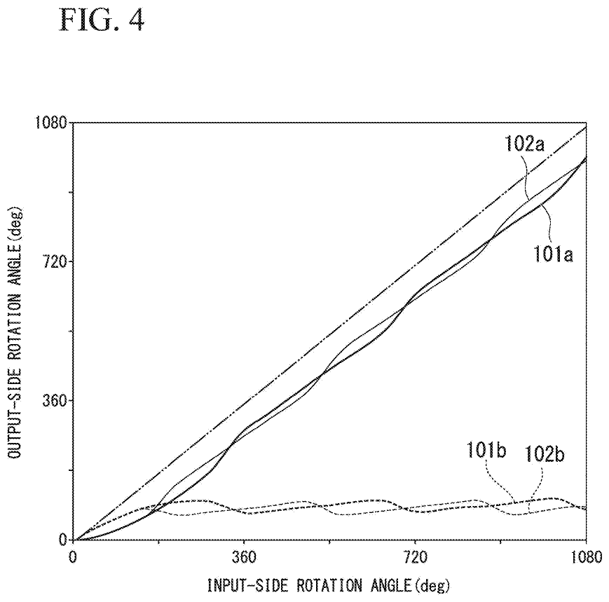

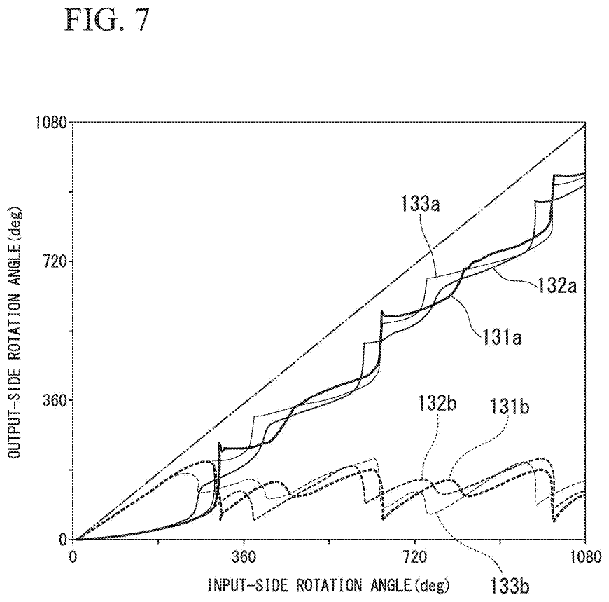

FIG. 4 to FIG. 6 are graphs showing the test results of the single wires of Examples 1 to 3. FIG. 7 to FIG. 9 are graphs showing the test results of the single wire wires of Comparison Examples 1 to 3.

In each graph, the horizontal axis represents the input side rotation angle (deg) and the vertical axis represents the output side rotation angle (deg).

Here, the “input side rotation angle” refers to a rotation angle based on the drive data of the grasping portion 51a.

The “output-side rotation angle” refers to a measured value regarding the rotation angle of the second end E2 of the single wire W. However, the solid line with the subscript “a” attached to the numeral reference indicates the actually measured value of the rotation angle (hereinafter referred to as an output value), while the broken line (the subscript “b” is attached to the numeral reference) indicates the magnitude of the difference between the output value and the input side rotation angle (=|output value−input side rotation angle|).

In each graph, the two-dot chain line indicates an ideal change (hereinafter referred to as an ideal line) in which the input side rotation angle and the rotation angle of the second end E2 completely match to each other. The broken line represents the deviation amount of the output value (solid line in the figure) from the ideal line in the vertical axis direction.

As the rotation transmission characteristic, it is more preferable that the deviation from the ideal line is smaller.

For example, it is more preferable that the change in the output value in the steady rotation stage is closer to a straight line parallel to the ideal line (with higher linearity). It is more preferable that the change from the average straight line in the change of the output value is smooth and the change range is small.

For example, it is more preferable that the input side rotation angle (hereinafter referred to as the transition angle) from the initial stage to the steady rotation stage is smaller. In this case, it is possible to pass through the initial stage in a short time.

Even if the transition angle is large, if the linearity in the steady rotation stage is high, it is considered that the operability is good. However, according to the study by the present inventor, when the transition angle is large, the above-mentioned deviation amount in the steady rotation stage tends to increase and the linearity of the output value tends to decrease.

In the evaluation of the rotation transmission characteristics, when the linearity of the change in the output value was high and the amount of deviation from the ideal line was small, it was judged to be “very good” (very good, “⊚” as shown in [Table 1]). When the linearity of the change in the output value was within the acceptable range and the amount of deviation from the ideal line was large, it was judged as “good” (good, “◯” as shown in [Table 1]). When the linearity of the change of the output value was out of the acceptable range, it was judged as “not acceptable” (no good, “x” as shown in [Table 1]). In particular, when a stepped change (popping behavior) was observed in the output value, it was judged as “not acceptable”.

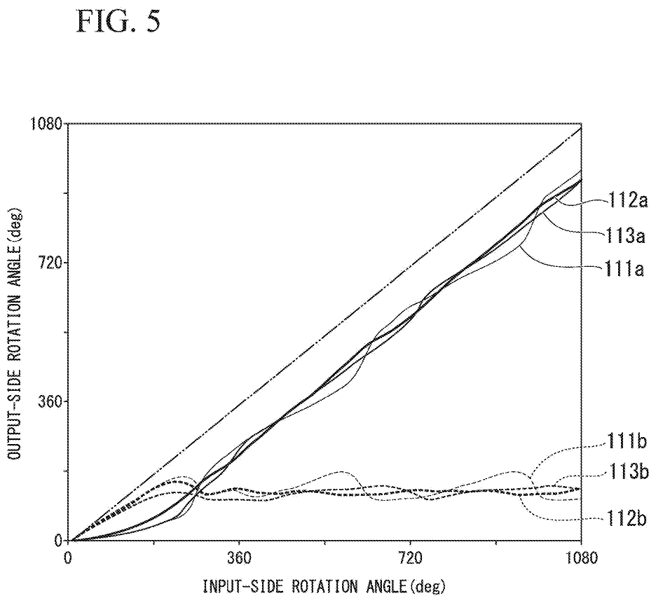

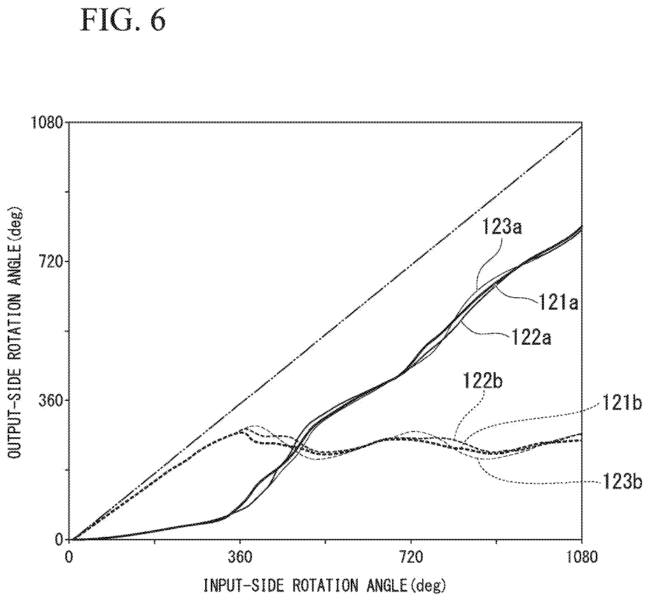

The test results of Examples 1 to 3 are shown in FIG. 4 to FIG. 6. FIG. 4 shows the test results of the two single wires 1. FIG. 5 and FIG. 6 show the test results of the three single wires 1.

In FIG. 4, as shown in the curve 101a and the curve 102a, each output value in Example 1 gradually increased in the initial stage. The initial stage was finished when the input side rotation angle was about 150 degrees.

Thereafter, the output value showed a substantially linear change following the change in the input side rotation angle. The output value changed slightly from the straight line parallel to the ideal line; however, the change was smooth and the change amount was small.

In the steady rotation stage, the curve 101b and the curve 102b were substantially constant around about 70 degrees.

The rotation transmission characteristic of Example 1 was determined to be “very good”.

When the clip 2 was rotated by using the treatment tool 10 using the single wire 1 for the treatment tool of Example 1, the operability was very good.

In FIG. 5, as shown by the curve 111a, the curve 112a, and the curve 113a, each output value in Example 2 gradually increased in the initial stage. The initial stage was finished when the input side rotation angle was about 250 degrees.

Thereafter, the output value showed a substantially linear change following the change in the input side rotation angle. The output value changed slightly from a straight line parallel to the ideal line; however, the change was smooth and the change amount was small. However, the deviation amount was slightly larger than that of Example 1, and there were some samples such as the curve 111a in which the change was slightly large.

In the steady rotation stage, the curve 111b, the curve 112b, and the curve 113b were substantially constant around about 120 degrees.

The rotation transmission characteristic of Example 2 was determined to be “very good”.

When the clip 2 was rotated by using the treatment tool 10 using the single wire 1 for the treatment tool of Example 2, the operability was very good.

In FIG. 6, as shown in the curve 121a, the curve 122a, and the curve 123a, each output value in Example 3 gradually increased in the initial stage. The initial stage was finished when the input side rotation angle was about 290 degrees.

Thereafter, the output value showed a substantially linear change following the change in the input side rotation angle. The output value changed from the straight line parallel to the ideal line; however, the change was smooth. However, the deviation amount and the change amount were larger than those of Examples 1 and 2.

Thereafter, the output value showed a substantially linear change following the change in the input side rotation angle. Therefore, the curve 121b, the curve 122b, and the curve 123b were substantially constant around about 240 degrees.

The rotation transmission characteristic of Example 3 was determined to be “good”.

When the clip 2 was rotated using the treatment tool 10 using the single wire 1 for the treatment tool of Example 3, the operation could be performed without any problem.

The test results of Comparison Examples 1 to 3 are shown in FIGS. 7 to 9, respectively. FIGS. 7-9 show the test results for each of the three single wires.

In FIG. 7, as shown in the curves 131a, 132a and 133a, each output value in Comparison Example 1 gradually increased in the initial stage and then sharply increased. The initial stage was finished when the input side rotation angle was about 220 degrees.

Thereafter, the output value changed significantly from the straight line parallel to the ideal line in a stepwise manner.

The transition angle and the average deviation amount (see curves 131b, 132b, and 133b) were both smaller than those in Example 3; however, they changed significantly in a stepwise manner at the steady rotation stage. It is considered difficult to adjust the rotation of the posture of the clip when it is assembled into the treatment tool.

Accordingly, the rotation transmission characteristic of Comparison Example 1 was determined to be “not acceptable”.

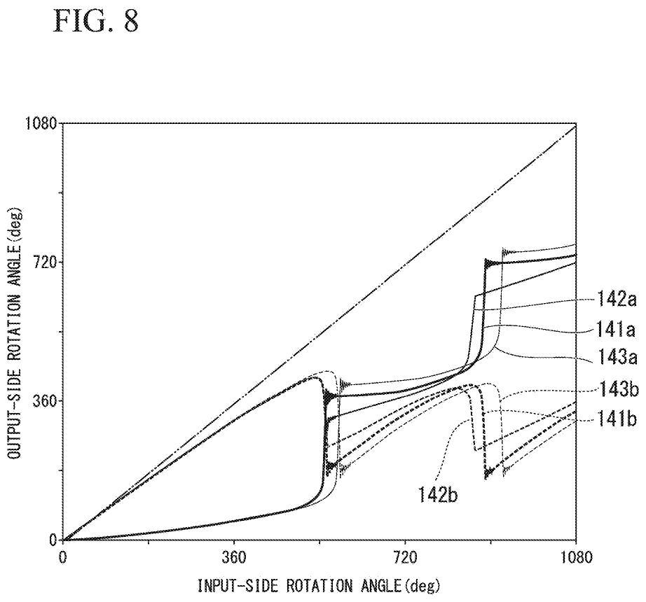

In FIG. 8, as shown in the curves 141a, 142a, and 143a, each output value in Comparison Example 2 gradually increased and then sharply increased in the initial stage. The initial stage was completed when the input side rotation angle was about 560 degrees.

Thereafter, the output value changed significantly from the straight line parallel to the ideal line in a stepwise manner. After the output value increased sharply, it was accompanied by vibration.

As shown in the curves 141b, 142b and 143b, the average deviation amount was also large.

Accordingly, the rotation transmission characteristic of Comparison Example 2 was determined to be “not acceptable”.

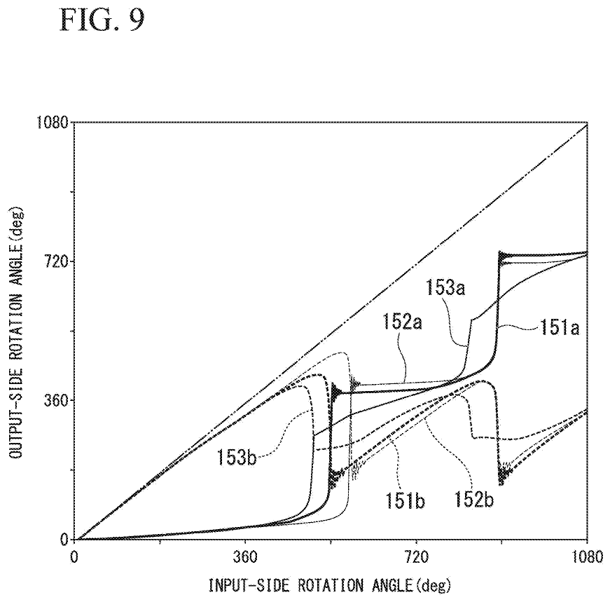

In FIG. 9, as shown in the curves 151a, 152a, 153a and curves 151b, 152b, 153b, the output value and the deviation amount of Comparison Example 3 showed the same changes as those in Comparison Example 2. Accordingly, the rotation transmission characteristic of Comparison Example 3 was determined to be “not acceptable”.

When the clip 2 was rotated by using the treatment tool 10 using the single wire for the treatment tools of Comparison Examples 1 to 3, it is impossible to adjust the clip 2 to the desired rotation position.

Since Comparison Examples 1 to 3 did not have the characteristic values required for the single wire according to the present disclosure, it is considered that the rotational transmission characteristics were poor.

Although each preferred embodiment of the present invention has been described above together with each embodiment, the present invention is not limited to this embodiment and each embodiment. Configurations can be added, omitted, replaced, and other modifications without departing from the spirit of the present invention.

Further, the present invention is not limited by the above description and is limited only by the appended claims.

What is claimed is:

1. A single wire for a medical treatment tool, comprising:
a main body portion formed from a material having an elastic limit stress equal to or more than 1400 MPa, a 0.2% yield strength equal to or more than 2000 MPa, and a breaking stress equal to or more than 2100 MPa; and
a locking portion formed at a distal end portion of the main body portion and being configured to lock the main body portion to the medical treatment tool.

2. The single wire for a medical treatment tool according to claim 1, wherein the material forming the main body portion further has an elastic limit elongation equal to or more than 1.0%, and a breaking elongation equal to or more than 3.0%.

3. The single wire for a medical treatment tool according to claim 1, wherein the main body portion has a diameter equal to or less than 0.5 mm.

4. The single wire for a medical treatment tool according to claim 1, wherein the medical treatment tool is either of a clip, a pair of grasping forceps, a pair of biopsy forceps, or a papillotome knife.

5. The single wire for a medical treatment tool according to claim 1, wherein the main body portion of the single wire is formed by stainless steel that is reformed by at least one of a straightening process or a heat treatment.

6. The single wire for a medical treatment tool according to claim 5, wherein the stainless steel contains chromium in a mass ratio equal to or more than 16% and nickel in a mass ratio equal to or more than 6%.

7. The single wire for a medical treatment tool according to claim 5, wherein the stainless steel is formed from at least one of a group consisting from SUS301, SUS304, and SUS631.

8. The single wire for a medical treatment tool according to claim 1, wherein the main body portion of the single wire is formed by stainless steel that is reformed by a heat treatment after a straightening process.

9. The single wire for a medical treatment tool according to claim 8, wherein the stainless steel contains chromium in a mass ratio equal to or more than 16% and nickel in a mass ratio equal to or more than 6%.

10. The single wire for a medical treatment tool according to claim 8, wherein the stainless steel is formed from at least one of a group consisting from SUS301, SUS304, and SUS631.

11. A medical treatment tool, comprising:
the single wire for a medical treatment tool according to claim 1.

12. A single wire for a medical treatment tool including a distal end function portion for performing medical treatment on living tissues, the single wire for a medical treatment tool comprising:
a distal end portion connected to the function portion; and
a proximal end portion,
wherein the single wire for a medical treatment tool is configured to transmit a rotation of the proximal end portion to the distal end portion, and
the single wire is formed from a material having an elastic limit stress equal to or more than 1400 MPa, a 0.2% yield strength equal to or more than 2000 MPa, and a breaking stress equal to or more than 2100 MPa.

13. A single wire for a medical treatment tool including a distal end function portion for performing medical treatment on living tissues, the single wire for a medical treatment tool comprising:
a distal end portion connected to the function portion,
wherein a distal end portion of the single wire for a medical treatment tool is connected to the distal end function portion, and
the single wire is formed from a material having an elastic limit stress equal to or more than 1400 MPa, a 0.2% yield strength equal to or more than 2000 MPa, and a breaking stress equal to or more than 2100 MPa.
